(19) United States
(12) Patent Application Publication
(10) Pub. No.: US 2022/0226406 A1
(43) Pub. Date: Jul. 21, 2022

(54) COMPOSITION CONTAINING EXTRACT OF CANNABIS SATIVA FOR PREVENTING OR TREATING METABOLIC SYNDROME-RELATED DISEASE

(71) Applicant: FAMENITY CO., LTD., Uiwang-si (KR)

(72) Inventors: Tae Wan Kim, Gwacheon-si (KR); Tae Joon Kim, Gwacheon-si (KR)

(21) Appl. No.: 17/460,257

(22) Filed: Aug. 29, 2021

(30) Foreign Application Priority Data
Jan. 18, 2021 (KR) 10-2021-0006862

(51) Int. Cl. A61K 36/185; A61K 31/05; A61K 31/01; A23L 33/105

(52) U.S. Cl. CPC A61K 36/185; A23L 33/105; A61K 31/01; A61K 31/05

(57) ABSTRACT

The present disclosure relate to a composition for preventing and treating metabolic disease containing a Cannabis sativa extract, and may provide a composition for preventing and treating metabolic disease, which contains an extract of the natural product Cannabis sativa, and thus has little or no side effects when taken or administered, and has an excellent effect of preventing or treating metabolic syndrome by reducing body weight, adipose tissue, blood glucose, triglyceride and cholesterol levels through promotion of AMPK activity and inhibition of the activity of the lipogenic transcription factor SREBP-1c.

### CROSS-REFERENCE TO RELATED APPLICATION

This application claims priority from and the benefit of Korean Patent Application No. 10-2021-0006862 filed on Jan. 18, 2021, which is hereby incorporated by reference for all purposes as if set forth herein.

### BACKGROUND

### Field

The present disclosure relate to a composition for preventing and treating metabolic disease containing a Cannabis sativa extract. More specifically, the present disclosure provides a composition for preventing and treating metabolic disease, which contains an extract of the natural product Cannabis sativa, and thus has little or no side effects when taken or administered, and has an excellent effect of preventing or treating metabolic disease by reducing body weight, adipose tissue, blood glucose, triglyceride and cholesterol levels through promotion of AMPK activity and inhibition of the activity of the lipogenic transcription factor SREBP-1c.

### Discussion of the Background

The prevalence of various lifestyle-related diseases and chronic degenerative diseases, which result from excessive nutrition, environmental pollution, lack of exercise, and increased stress due to the improvement of the economic level, has emerged as a major problem in our society. Among these diseases, metabolic syndrome is a disease that has attracted the most attention in recent years.

The term “metabolic syndrome” began to be used in the late 1950s, has been commonly used since the late 1970s, and is also referred to as metabolic disease. In 1988, Reaven proposed insulin resistance as a cause of metabolic syndrome, and defined a variety of abnormal symptoms, that is, abdominal obesity, dyslipidemia, high blood pressure, and fasting hyperglycemia, as “Syndrome X”. Metabolic syndrome is determined by genetic and environmental factors, and is also affected by factors such as age, smoking, drinking, diet, and physical activity. Metabolic syndrome has been reported as a major risk factor for diabetes mellitus and cardiovascular diseases.

The main symptoms of the metabolic syndrome include diabetes caused by abnormal blood glucose metabolism, obesity, increased triglycerides or dyslipidemia caused by abnormal lipid metabolism, hypertension caused by high density cholesterol and increased sodium levels, and gout caused by increased uric acid, and it has been reported that various adult diseases such as stroke, arteriosclerosis and heart disease are also caused by metabolic syndrome. A recent survey estimates that about a quarter of American adults have metabolic syndrome. In addition, it has been reported that 15 to 20% of Koreans in their 30s and 30 to 40% of Koreans over 40 years old have metabolic syndrome, and these patients with metabolic syndrome continue to increase rapidly.

Diabetes, which is a representative disease of metabolic syndrome, is a disease occurring when hyperglycemia is maintained because blood glucose levels are not properly controlled due to abnormalities in insulin secreting cells (beta-cells) or abnormalities in insulin action.

Meanwhile, although obesity has become a social issue in terms of aesthetics in appearance, it is the most serious problem of obesity that obesity can actually lead to serious health risks such as metabolic disease complications such as diabetes and hypertension. A symptom related to the pathological state of obesity is a systemic chronic inflammation that appears in obese individuals.

Inflammatory response is one of the immune mechanisms occurring in the body, and is an important response that protects the body from invasion of external pathogens or viruses when it occurs locally. However, when this inflammatory response is systemically and chronically overactive due to the breakdown of the balance of immune responses in the body, it causes a disorder in the metabolism occurring in the body.

In particular, chronic inflammatory response caused by obesity has been found to be the cause of various metabolic diseases such as diabetes, cardiovascular disease and arteriosclerosis, and is also the most important factor defining obesity as a disease. Obesity is merely a cosmetic problem without the onset of secondary metabolic diseases due to chronic inflammatory response, and recently, the World Health Organization has also defined obesity as a disease for reasons of chronic inflammatory responses that can cause secondary metabolic diseases (such as diabetes) that significantly reduce the quality of life.

When obesity is induced, it results in abnormalities in visceral adipose tissue, and adipose tissue secretes endocrine factors such as adiponectin, plasminogen activator inhibitor, monocyte chemotactic protein-1 (MCP-1), tumor necrosis factor (TNF-α) and leptin.

In particular, when monocyte chemotactic protein-1 (MCP-1) and tumor necrosis factor (TNF-α) are excessively secreted, immune cells such as macrophages infiltrate adipose tissue and increase the expression of inflammatory cytokines such as interleukin-6 (IL-6) in addition to monocyte chemotactic protein-1 (MCP-1) and tumor necrosis factor (TNF-α). As a result, chronic inflammation of the adipose tissue occurs, and the chronic inflammatory response decreases insulin sensitivity and causes glucose tolerance, leading to diabetes disease.

This is a symptom that occurs because wastes accumulated in the human body due to the inability to release metabolic waste products and toxins that occur due to poorly balanced metabolism cause the loss of function of each organ of the human body. This symptom develops into metabolic syndrome, also known as insulin resistance syndrome. The metabolic syndrome, in turn, causes damage to the coronary artery, causing heart disease or stroke, or reduces the ability of the kidney to remove salt, causing hypertension, increases triglyceride levels, causing cardiovascular disease, and increases the risk of blood clotting. In addition, this metabolic syndrome is known to cause damage to the eyes, kidneys and nerves due to decreased insulin production in type 2 diabetes.

Accordingly, in consideration of problems related to various metabolic syndrome-related diseases, increased efforts have been made to develop natural substances which have no side effects and are effective against metabolic syndrome-related diseases, including hyperlipidemia, diabetes and liver disease. It is expected that these natural substances can provide an anti-obesity effect by suppressing adipose tissue hypertrophy and inflammatory response, and as a result, exhibit anti-metabolic syndrome effects.

### PRIOR ART DOCUMENTS

### Patent Documents

(Patent Document 1) KR 10-2019-0048996 A

(Patent Document 2) KR 10-2019-0048997 A

### SUMMARY

An object of the present disclosure is to provide a composition for preventing and treating metabolic disease, which contains an extract of the natural product Cannabis sativa, and thus has little or no side effects when taken or administered.

Another object of the present disclosure is to provide a composition for preventing and treating metabolic disease, which has an excellent effect of preventing or treating metabolic disease by reducing body weight, adipose tissue, blood glucose, triglyceride and cholesterol levels through promotion of AMPK activity and inhibition of the activity of the lipogenic transcription factor SREBP-1c and the expression of fatty acid synthase.

To achieve the above objects, a composition for preventing and treating metabolic disease according to one embodiment of the present disclosure contains a Cannabis sativa extract as an active ingredient.

The Cannabis sativa extract contains cannabidiol and terpene.

The metabolic disease includes a disease selected from the group consisting of obesity, diabetes, hyperlipidemia, hypertension, hypercholesterolemia, hyperinsulinemia, arteriosclerosis and fatty liver.

The composition inhibits the activity of a lipogenic transcription factor by promoting AMPK activity.

The lipogenic transcription factor is SREBP-1c (sterol regulatory element-binding protein-1c).

A food composition for preventing metabolic disease according to another embodiment of the present disclosure is produced to contain the above-described composition.

A pharmaceutical composition for treating metabolic disease according to still another embodiment of the present disclosure is produced to contain the above-described composition.

Hereinafter, the present disclosure will be described in more detail.

As used herein, the term “preventing” refers to any action of suppressing or delaying metabolic syndrome-related diseases, including obesity, hyperlipidemia, hypertension, hypercholesterolemia, hyperinsulinemia, arteriosclerosis and fatty liver, by administering the composition according to the present disclosure.

As used herein, the term “treating” refers to any action of alleviating or beneficially changing the above-described diseases by administering the composition according to the present disclosure.

As used herein, the term “metabolic disease” refers to a variety of diseases that occur due to problems in metabolism, and is also referred to as a metabolic syndrome-related disease.

Although the metabolic syndrome-related disease in the present disclosure include, without limitation, diseases which may be treated or prevented with the Cannabis sativa extract as an active ingredient, it may be, for example, one or more diseases selected from the group consisting of obesity, diabetes, hyperlipidemia, hypertension, hypercholesterolemia, hyperinsulinemia, arteriosclerosis and fatty liver.

As used herein, the term “extract” not only means a crude extract that is commonly used in the art as described above, but also includes, in a broad sense, a fraction obtained by fractionating the extract. That is, the term “extract” includes not only an extract obtained using an extraction solvent, but also one obtained by additionally applying a purification process to the extract. For example, the term “extract” as used herein also include a fraction obtained by passing the extract through an ultrafiltration membrane having a certain molecular weight cut-off value, and fractions obtained by additionally performing various purification processes, such as separation by various chromatography systems (manufactured for separation according to size, charge, hydrophobicity or affinity).

A composition for preventing and treating metabolic disease according to one embodiment of the present disclosure contains a Cannabis sativa extract as an active ingredient.

Cannabis sativa is an annual plant belonging to the genus Cannabis of the family Cannabaceae, and is flowing plant species including three different subspecies: C. sativa, C. indica, and C. ruderalis.

As far as is known, about 400 compounds have been found in Cannabis sativa, and most of them are cannabinoids, terpenes, and phenolic compounds. Among them, cannabinoids are known as representative active ingredients of Cannabis sativa. About 90 kinds of cannabinoids have been identified to date, and a number of ingredients found only in Cannabis sativa are also known. Cannabinol (CBN) was isolated from Cannabis sativa in 1899, but it was later found that the cannabinol was not a single compound. Since cannabidiol (CBD) and tetrahydrocannabinol (THC), which are pure compounds, were isolated from Cannabis sativa in the 1930s, studies on the components of Cannabis sativa have been more actively conducted.

Efforts to develop drugs using specific components of Cannabis sativa have also been continued, and among these specific components, THC and CBD, which are major compounds of Cannabis sativa, have attracted the most attention for therapeutic purposes. Some studies indicated that CBD has no phrenotropic action and is effective in reducing pain and controlling epileptic seizures.

In addition, more than 100 terpene-based compounds that play a role in the flavor and taste of Cannabis sativa were also found in Cannabis sativa, and are present as various monoterpenoids and sesquiterpenoids. Terpenes have been found to be related to various pharmacological actions such as anti-inflammatory action, but studies on terpene compounds extracted from Cannabis sativa are still insufficient compared to THC.

The Cannabis sativa extract contains cannabidiol and terpene.

Cannabidiol (CBD) is one of the main components of Cannabis and is a compound that is much comparable with tetrahydrocannabinol (THC). In the case of Korea, cannabidiol has been designated as a narcotic, and thus many studies thereon have not been conducted, but in foreign countries, cannabidiol has been actually used as a medical drug for relieving symptoms such as pain, memory disorder, and anxiety, and active studies thereon have been conducted.

Tetrahydrocannabinol (THC) is a major psychotropic component of the Cannabis sativa plant, and THC is psychotropic only in a decarboxylated state. THC has a structure similar to that of CBD, but it is known that THC induces excitement and has an apoptotic effect in some cancers, whereas CBD has been less studied compared to THC and does not induce excitement.

The most well-studied cannabinoids include tetrahydrocannabinol (THC), cannabidiol (CBD) and cannabinol (CBN). Other cannabinoids include, for example, cannabichromene (CBC), cannabigerol (CBG), cannabinidiol (CBND), cannabicyclol (CBL), cannabivarin (CBV), tetrahydrocannabivarin (THCV), cannabidivarin (CBDV), cannabichromevarin (CBCV), cannabigerovarin (CBGV), and cannabigerol monomethyl ether (CBGM).

Terpenes are known to exhibit better effects when acting together with cannabinoids such as CBD and THC, and may improve the uptake of cannabinoids, overcome the bacterial defense mechanism and minimize side effects.

Cannabis sativa has been used in various ways in the past depending on parts thereof. Specifically, it is known that the leaf of Cannabis sativa has the effect of killing roundworms, and that, when hair is washed with water obtained by boiling the leaf of Cannabis sativa, the hair grows long and becomes abundant. In addition, the leaf of Cannabis sativa was used against asthma or old cough or roundworms, or as an analgesic, anesthetic or diuretic agent. There is a record that the root of Cannabis sativa was used for the treatment of difficult delivery and “placenta not coming out”, the removal of extravasated blood, and the treatment of urolithiasis, and was taken as a water decoction. There is a record that the shell of Cannabis sativa was used for the treatment of bruises and fever-type intestinal pain, and the flower of Cannabis sativa was used for paralysis symptoms and itching. The flower spike of Cannabis sativa was used for difficulty delivery, constipation, gout, manic depressive psychosis, insomnia, and the like. In particular, the seeds of Cannabis sativa are rich in L-arginine, and thus may exhibit a tension relieving effect by releasing muscle tension, and cannabinoids, which are the unique components of Cannabis sativa, are effective in pain relief and tension relief.

The metabolic disease includes a disease selected from the group consisting of obesity, diabetes, hyperlipidemia, hypertension, hypercholesterolemia, hyperinsulinemia, arteriosclerosis and fatty liver.

The metabolic disease refers to a condition or disease that is closely related to obesity or is caused by obesity.

The obesity refers to a condition in which adipocytes proliferate and differentiate in the body due to metabolic disorders, and hence fat is excessively accumulated in the body. Obesity may cause related complications including metabolic syndrome accompanied by hypertension, diabetes and dyslipidemia.

The diabetes refers to a disease that occurs when the secretion of insulin is deficient or the action and function of insulin is insufficient. This disease causes abnormal elevation of glucose concentration in the liver or blood due to excessive degradation of glycogen, protein and lipids, which may result in glycosuria and ketonuria. Diabetes may also cause a morbid condition such as hemoconcentration, circulatory disturbance, or renal disorders, which are induced by loss of electrolytes caused by metabolic abnormality of moisture and electrolytes. Insulin is secreted from β-cells of Langerhans islets present inside the pancreas, and insulin is secreted when the blood glucose concentration increases, whereas secretion of insulin is suppressed when the blood glucose concentration decreases, thereby regulating appropriate activities of energy sources. This disease is classified into insulin-dependent diabetes (Type I) and insulin-independent diabetes mellitus (Type II). Diagnosis of diabetes is generally possible by measurement of the blood glucose concentration and varies depending on the criteria. In general, humans are diagnosed with diabetes when normal glucose concentration in blood is 200 mg/dL or higher or when fasting glucose concentration in blood is 140 mg/dL or higher. Accordingly, diabetes may be treated or prevented by reducing the glucose concentration in the blood or the liver.

The hyperlipidemia refers to a condition or disease in which the concentrations of blood lipid components, particularly cholesterols and triglycerides, are higher than the normal levels. In addition, hyperlipidemia is used in a broad sense including all conditions in which it is required to lower the blood lipid concentration. Hyperlipidemia is characterized by increased concentrations of blood lipid components, especially cholesterols and triglycerides. Generally, a blood cholesterol concentration higher than 240 mg/dl or a blood triglyceride concentration of 200 mg/dl or higher is referred to as hyperlipidemia. Hyperlipidemia may be caused by a genetic predisposition, obesity, dietary habits, diabetes, nephrotic syndrome, or hypothyroidism.

The hypertension refers to a condition in which the blood pressure of the arteries is chronically high. Hypertension also refers to a case in which an adult 18 years of age or older has a systolic blood pressure of 140 mmHg or more or a diastolic blood pressure of 90 mmHg or more. Hypertension may also be caused by obesity or the like.

The hypercholesterolemia refers to a condition in which the serum cholesterol concentration is 220 to 250 mg/dL or more. Hypercholesterolemia is a disease that is likely to lead to atherosclerotic disease. Hypercholesterolemia may be classified into primary and secondary. Primary hypercholesterolemia is a dominant genetic disorder that is caused by decreased function of LDL receptors in the liver and other cell membranes, and secondary hypercholesterolemia is caused by obesity, nephrosis, hypothyroidism, obstructive jaundice, and diabetes.

The hyperinsulinemia is a condition in which blood insulin levels are high. Hyperinsulinemia is a disease which is associated with obesity or diabetes and enhances activation of sympathetic nerves or promotes sodium uptake in the kidney.

The arteriosclerosis refers to a condition or disease in which blood circulation to organs and tissues in the body is lowered due to the thickening and decreased elasticity of the arterial wall. In addition, the arteriosclerosis is meant to include “atherosclerosis” which means a condition or disease in which blood circulation is lowered by narrowing of the lumen due to plaques formed by deposition of other substances such as fat and cholesterol on the inner wall of the artery. Arteriosclerosis may occur anywhere in the body. If arteriosclerosis occurs in the blood vessels in the heart, it may cause coronary artery diseases such as angina pectoris and myocardial infarction, and if arteriosclerosis occurs in the brain, it may cause cerebral infarction, and if arteriosclerosis occurs in the kidney, it may cause kidney failure and the like.

The fatty liver refers to a condition or disease in which fat is excessively accumulated in liver cells due to a hepatic fat metabolism disorder. Most of the fat accumulated in fatty liver is triglyceride, and fatty liver may be broadly divided into alcoholic fatty liver caused by heavy drinking, and non-alcoholic fatty liver caused by obesity, diabetes, hyperlipidemia or drugs. Alcoholic fatty liver occurs because excessive intake of alcohol promotes fat synthesis in the liver and interferes with normal energy metabolism.

The composition for preventing and treating metabolic disease according to the present disclosure exhibits the effect of ameliorating, preventing and treating metabolic disease caused by body weight or body fat gain due to the above-described various factors, more specifically, an obesity or metabolic disease induced by a high-fat diet.

The composition inhibits the activity of a lipogenic transcription factor by promoting AMPK activity.

AMP-activated protein kinase (AMPK) is an enzyme that is mainly expressed in tissues such as liver, muscle and adipose and plays an important role in intracellular energy metabolism. AMPK is activated by decreased ATP levels and increased AMP levels due to intracellular energy depletion, and activation of AMPK functions to inhibit the synthesis of intracellular fat and promote the degradation of intracellular fat in the human body. Accordingly, AMPK is well known as a therapeutic target against metabolic diseases such as obesity, diabetes, fatty liver, and hyperlipidemia.

Substrate proteins known to be phosphorylated by AMPK include AMPK, acetyl-CoA carboxylase (ACC), and SREBP-1c (sterol regulatory element-binding protein-1c).

In particular, SREBP (sterol regulatory element-binding protein) is an important transcriptional activator that induces the synthesis of cholesterol and fatty acids in the liver and adipocytes by expressing enzymes related to the biosynthetic pathway of fatty acids and cholesterol. SREBP is classified into three isoforms: SREBP-1a, SREBP-1c, and SREBP-2. Among them, SREBP-1c is most often expressed in tissues such as fat, liver, and muscle, and it is known that ACC1 (acetyl-CoA carboxylase 1), FAS (fatty acid synthatase), SCD1 (stearoyl-CoA desaturase 1) and SREBP-1c, which are major enzymes involved in the synthesis of fat, function as transcription factors that are expressed by themselves.

In addition, it is known that phosphorylation of SREBP-1c by AMPK reduces the activity of SREBP-1c.

The composition for preventing and treating metabolic disease containing a Cannabis sativa extract as an active ingredient according to the present disclosure may exhibit the effect of preventing and treating metabolic disease by inhibiting the lipogenic transcription factor SREBP-1c (sterol regulatory element-binding protein-1c).

The Cannabis sativa extract is obtained by extraction with an extraction solvent selected from the group consisting of water, a Ci to C6 lower alcohol, and a mixture thereof.

Specifically, the Cannabis sativa extract as a natural extract may be obtained by a method including steps of: crushing a natural product to obtain a sample; leaching the sample with an organic solvent; drying the leached sample; re-leaching the dried sample with an organic solvent; drying the re-leached sample; leaching the dried sample with water; and leaching.

The natural extract obtained by extraction with the organic solvent may be further subjected to a fractionation step using an organic solvent.

The extraction solvent may be used in an amount equal to 2 to 50 times, more specifically 2 to 20 times, the weight of the sample. For leaching and extraction, the sample may be left to stand in the extraction solvent for 1 to 72 hours, more specifically 24 to 48 hours.

The extract may be prepared in a powder state by additional processes such as reduced pressure distillation and freeze drying or spray drying, and is obtained by an extraction method selected from the group consisting of a solvent extraction method, an ultrasonic extraction method, a reflux extraction method, a leaching method, a fermentation method, and a processing method.

The ultrasonic extraction method is performed by extraction using water or a 50 to 100% alcohol having 1 to 6 carbon atoms as an extraction solvent at 30 to 50° C. for 0.5 to 2.5 hours. Specifically, the ultrasonic extraction method includes is performed by extraction using water or a 70 to 80% alcohol having 1 to 6 carbon atoms as an extraction solvent at 40 to 50° C. for 1 to 2.5 hours.

The reflux extraction method is performed by refluxing 10 to 30 g of the crushed natural product in 100 mL of water or a 50 to 100% alcohol having 1 to 6 carbon atoms for 1 to 3 hours. More specifically, the reflux extraction method is performed by refluxing 10 to 20 g of the crushed natural product in 100 mL of water or a 70 to 90% alcohol having 1 to 4 carbon atoms for 1 to 2 hours.

The leaching method is performed by using water or a 50 to 100% alcohol having 1 to 6 carbon atoms as an extraction solvent at 15 to 30° C. for 24 to 72 hours. More specifically, the leaching method is performed by using water or a 70 to 80% alcohol having 1 to 6 carbon atoms as an extraction solvent at 20 to 25° C. for 30 to 54 hours.

After extraction, the extract may be fractionated sequentially using fresh fractionation solvents. The fractionation solvent that is used for fractionation of the extract is any one or more selected from the group consisting of water, hexane, butanol, ethyl acetic acid, ethyl acetate, methylene chloride, and mixtures thereof. Preferably, the fractionation solvent is ethyl acetate or methylene chloride.

Preferably, the composition for preventing and treating metabolic disease containing a Cannabis sativa extract as an active ingredient may additionally contain an extract of Polypogon monspeliensis, an extract of Artemisia sylvatica Maxim., and an extract of Aster fastigiatus Fisch.

The Polypogon monspeliensis is a weed belonging to the genus Polypogon of the family Pooideae. Polypogon monspeliensis is native to southern Europe, but now spreads all over the world, and is an annual grass growing to a height of 5 cm to 1 m.

The Artemisia sylvatica Maxim. is a perennial dicotyledonous plant belonging to the family Asteraceae of the order Campanulales, and grows in mountain forests. Leaves from the roots of Artemisia sylvatica Maxim. remain until flowering, spread in a rose flower shape, have an egg shape or a long oval shape, and have pointed ends. The leaf thereof is 11 to 20 cm in length and 7 to 9.5 cm in width, and the surface thereof has slightly curly hairs. The backside of the leaf has cobweb-like hairs, and the leaf has pointed teeth at the edges thereof. Leaves from the stems are similar to but different in size from the leaves from the roots.

The Aster fastigiatus Fisch. is a perennial herb distributed in Korea, China, Japan, and Russia. In Korea, Aster fastigiatus Fisch. is distributed throughout the country, and grows to about 30 to 100 cm in height. The stem thereof is 30 to 100 cm in height, is upright, has a vertical ridge, and the branches from the upper part thereof are arranged in a corymbose manner, and rough hairs grow densely on the stem. The flower blooms in August through October, is 7 to 9 mm in diameter, grows in a corymb inflorescence at the end of the main stem, and has a flower stalk length of 3 to 8 mm. The involucre is tubular, has a length of 4 mm and a width of 5 mm. The bracts are arranged in 4 rows, lanceolate and obtuse, and have many hairs, and the inner Involucre is 1.5 mm in length. The ligulate flowers are arranged in one row and white, and the corolla is 5 to 6.5 mm in length and 1 mm in width. The leaves remain until the leaves that came out at first flower. The leaves are linear-lanceolate, narrow at both ends, 5 to 12 cm in length, and 4 to 15 mm in width. The lower part of the leaf is narrowed to become a petiole, and the backside of the leaf is whitish, and has pellucid dots and sericeous hairs. The leaf has sparse serrations at the margins thereof, is often rolled back, and as short hairs on the upper edge thereof. The cauline leaf becomes gradually smaller as it goes upward, and is linear-lanceolate or linear, and the backside thereof has densely sericeous hairs and pellucid dots, and the leaves of the inflorescence are 2 to 3 mm in length.

When the natural extracts are used in combination, they may exhibit a synergistic effect, and thus exhibit an excellent effect of suppressing body weight and body fat gain caused by a high-fat diet in a diet-induced obesity mouse model, thus exhibiting an effect of preventing and treating obesity and metabolic disease.

In addition, as the extract of Polypogon monspeliensis, the extract of Artemisia sylvatica Maxim., and the extract of Aster fastigiatus Fisch. are additionally contained, it is possible to provide a composition having excellent palatability by neutralizing the unique taste and flavor of the Cannabis sativa extract.

Preferably, the composition of the present disclosure may contain, based on 100 parts by weight of Cannabis sativa extract, 20 to 40 parts by weight of the extract of Polypogon monspeliensis, 20 to 40 parts by weight of the extract of Artemisia sylvatica Maxim., and 20 to 40 parts by weight of the extract of Aster fastigiatus Fisch.

When the extracts are used in combination in amounts within the above-described ranges, it is possible to provide a composition having excellent palatability while exhibiting an excellent effect of preventing and treating metabolic disease.

The composition for preventing and treating metabolic disease containing a Cannabis sativa extract as an active ingredient according to the present disclosure may be used in various applications.

A food composition for preventing metabolic disease according to another embodiment of the present disclosure is produced to contain the above-described composition.

As used herein, the term “functional food” refers to foods produced and processed using functional raw materials or ingredients beneficial to human health pursuant to Health Functional Foods Act No. 6727, and the term “functionality” means controlling nutrients for the structure or functions of the human body or providing beneficial effects to health purposes, such as physiological effects.

A pharmaceutical composition for treating metabolic disease according to still another embodiment of the present disclosure is produced to contain the above-described composition.

The dosage form of a medicament of the present disclosure may be preferred form selected depending on the method of use thereof, and specific examples of the dosage form include granules, powders, syrups, liquids, suspensions, decoctions, infusions, tablets, suppositories, injections, spirits, capsules, pills, and soft or hard gelatin capsules.

In addition, if necessary, the medicament of the present disclosure may further contain an excipient, a filler, an extender, a binder, a disintegrant, a lubricant, a preservative, an antioxidant, an isotonic agent, a buffer, a film-forming agent, a sweetening agent, a solubilizing agent, a base agent, a dispersing agent, a wetting agent, a suspending agent, a stabilizer, a colorant, a fragrance, etc. which are commonly used in the art.

In the manufacture of the medicament, the content of the composition for preventing and treating metabolic disease according to the present disclosure may vary depending on the form of the medicament, and the dosage thereof may be easily adjusted by those skilled in the art depending on the type of subject to be treated, the route of administration, the subject's weight, sex, and age, and the severity of the disease.

### BRIEF DESCRIPTION OF THE DRAWINGS

FIG. 1 shows the results of analyzing the effect of a composition according to one embodiment of the present disclosure on the target DNA-binding activity of SREBP-1c in comparison with metformin.

FIG. 2 shows the results of comparing mouse body weight and food intake between mice, to which a high-fat diet was administered and the composition according to one embodiment of the present disclosure was orally administered, and a control group.

FIG. 3 graphically shows the effect of the composition according to one embodiment of the present disclosure on blood glucose levels.

FIG. 4 graphically shows the effect of the composition according to one embodiment of the present disclosure on blood triglyceride levels.

FIG. 5 graphically shows the effect of the composition according to one embodiment of the present disclosure on blood LDL-cholesterol levels.

FIG. 6 graphically shows the effect of the composition according to one embodiment of the present disclosure on blood total cholesterol levels.

### DETAILED DESCRIPTION OF THE ILLUSTRATED EMBODIMENTS

Hereinafter, examples of the present disclosure will be described in detail so that those of ordinary skill in the art can easily carry out the present disclosure. However, the present disclosure may be embodied in a variety of different forms and is not limited to the examples described herein.

### Production Example 1: Production of Extracts

1. Production of Cannabis sativa Extract

Cannabis sativa including leaves and flowers was washed clean with running water, and then completely dried naturally. The dried Cannabis sativa was crushed with a mixer and then prepared into powder. 100 g of Cannabis sativa powder was immersed in 1,000 g of ethanol and eluted at 40° C. for 48 hours. Thereafter, the solid was removed by centrifugation, and the remaining supernatant was collected and filtered. The filtrate was subjected to a conventional concentration process under reduced pressure to obtain a Cannabis sativa extract (CE) containing cannabidiol and terpene at a concentration of 0.15 mg/ml.

2. Production of Other Natural Extracts

First, Polypogon monspeliensis was washed, dried and then crushed. The crushed Polypogon monspeliensis was added to a 60% ethanol and extracted for 2 hours. The extract was cooled and then filtered through Whatman filter paper. The filtrate was collected, thus producing a Polypogon monspeliensis extract (PE).

An Artemisia sylvatica Maxim. extract (AE) and an Aster fastigiatus Fisch extract (OE) were produced according to the same method as the method for producing the Polypogon monspeliensis extract (PE).

3. Production of Extract Mixtures

The Cannabis sativa extract (CE), the Polypogon monspeliensis extract (PE), the Artemisia sylvatica Maxim. extract (AE) and the Aster fastigiatus Fisch extract (OE) were mixed together as shown in Table 1 below to obtain extract mixtures.

TABLE 1

| | MT1 | MT2 | MT3 | MT4 | MT5 | MT6 |
|---|---|---|---|---|---|---|
| CE | 100 | 100 | 100 | 100 | 100 | 100 |
| PE | — | 10 | 20 | 30 | 40 | 50 |
| AE | — | 10 | 20 | 30 | 40 | 50 |
| OE | — | 10 | 50 | 30 | 40 | 50 |

(unit: parts by weight)

### Test Example 1: Cytotoxicity Test

To test the toxicity of each of the Cannabis sativa extract (CE) (MT1) and the extract mixtures (MT2 to MT6) produced in Production Example 1, differences in toxicity and side effects caused by administration of the extract mixtures in repeated-dose toxicity tests for rats were examined.

6-week-old male and female SD rats were divided into a plurality of groups, each consisting of 10 rats (5 male rats and 5 female rats), and each of the Cannabis sativa extract (CE) (MT1) and the extract mixtures (MT2 to MT6) was administered to the rats. Each of the Cannabis sativa extract and the extract mixtures was dissolved in a 0.5% methylene chloride (MC) solution and then administered orally once at the same time in the morning every day. This administration was repeated for 13 weeks. Each of the Cannabis sativa extract (CE) and the extract mixtures was administered once a day at a daily dose of 3.75 mg/kg to 5 mg/kg. Thereafter, mortality, general symptoms, weight changes, and feed and water intakes were observed.

As a result, no death occurred within the test period. In view of the above test results, it was confirmed that the Cannabis sativa extract (CE) (MT1) and the extract mixtures (MT2 to MT6) had no toxicity problem.

### Test Example 2: Effect of Cannabis sativa Extract on AMPK Activity

In order to examine the effect of each of the Cannabis sativa extract (CE) (MT1) and extract mixtures (MT2 to MT6), produced in Production Example 1, on the activity of AMPK, the following experiment was performed. AMPK, a serine/threonine kinase, is activated by decreased ATP levels and increased AMP levels due to intracellular energy depletion, and activation of AMPK inhibits the synthesis of intracellular fat and promotes the degradation of intracellular fat in the human body. Accordingly, AMPK is well known as a therapeutic target against metabolic diseases such as obesity, diabetes, fatty liver, and hyperlipidemia.

Substrate proteins known to be phosphorylated by AMPK include AMPK, acetyl-CoA carboxylase (ACC), and SREBP-1c (sterol regulatory element-binding protein-1c).

First, 3T3-L1 adipocytes were cultured, and each of the Cannabis sativa extract (CE) (MT1) and extract mixtures (MT2 to MT6) of the present disclosure was dissolved in DMSO (dimethyl sulfoxide) to a final concentration of 0.2%. The 3T3-L1 adipocytes were treated with each of the solutions. It was confirmed that 0.2% DMSO was not toxic to the cells. A control group was treated with 0.2% DMSO.

Thereafter, the cells cultured according to the experimental method were collected, lysed, and then placed in a 95-well plate, and the activity of AMPK in the cells was quantified using an AMPK assay kit (CycLex Co. Japan).

As a result, as shown in Table 2 below, it was confirmed that the AMPK activity in the cells treated with the Cannabis sativa extract (CE) (MT1) increased compared to that in the control group.

TABLE 2

| | Control | | | | | | |
|---|---|---|---|---|---|---|---|
| | (adipocyte) | MT1 | MT2 | MT3 | MT4 | MT5 | MT6 |
| AMPK activity | 1.000 ± | 1.344 ± | 1.473 ± | 1.509 ± | 1.772 ± | 1.867 ± | 1.589 ± |
| (mean ± | 0.236 | 0.003 | 0.042 | 0.861 | 0.132 | 0.208 | 0.048 |
| standard | | | | | | | |
| deviation) | | | | | | | |

Particularly, when comparing between the groups treated with the extract mixtures (MT2 to MT6), respectively, treatment with each of MT3 to MT5 showed the highest rate of AMPK activity promotion, and treatment with each of MT2 and MT6 showed the lowest rate of AMPK activity promotion. Thereby, it could be confirmed that the extract mixtures MT3 to MT5 of the present disclosure had the best effect on the promotion of AMPK activity.

### Test Example 3: Effect of Cannabis sativa Extract on SREBP-1c Activity

After confirming the AMPK activity promotion effect in Test Example 2, the following experiment was performed in order to examine the effect of each of the Cannabis sativa extract (CE) (MT1) and extract mixtures (MT2 to MT6) on the activity of SREBP-1c.

SREBP (sterol regulatory element-binding protein) is an important transcriptional activator that induces the synthesis of cholesterol and fatty acids in the liver and adipocytes by expressing enzymes related to the biosynthetic pathway of fatty acids and cholesterol, and is classified into three isoforms: SREBP-1a, SREBP-1c, and SREBP-2. Among them, SREBP-1c is most often expressed in tissues such as fat, liver, and muscle, and it is known that ACC1 (acetyl-CoA carboxylase 1), FAS (fatty acid synthatase), SCD1 (stearoyl-CoA desaturase 1) and SREBP-1c, which are major enzymes involved in the synthesis of fat, function as transcription factors that are expressed by themselves. In addition, it is known that phosphorylation of SREBP-1c by AMPK reduces the activity of SREBP-1c.

According to the above-described experimental method, 3T3-L1 adipocytes were treated with 1,000 μg/ml of each of the Cannabis sativa extract (CE) (MT1) and extract mixtures (MT2 to MT6) for 7 days. For comparison, 3T3-L1 adipocytes were treated with 1,000 μg/ml of metformin (MET), a representative AMPK promoter among commercially available pharmaceuticals. Thereafter, the target DNA (5′-TCACCTGA-3′)-binding activity of SREBP-1c in the cells was measured using a SREBP-1 transcription factor ELISA (Cayman Chemical Co. Ann Arbor, Mich., USA).

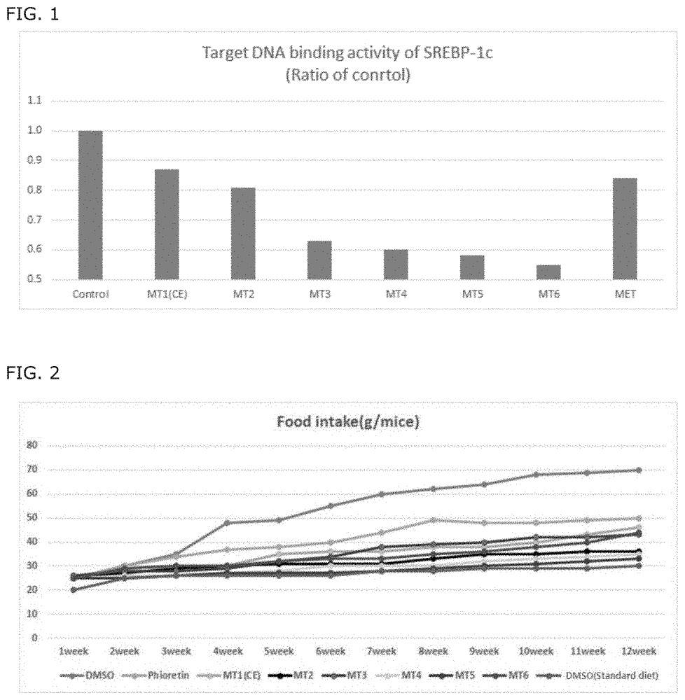

As a result, as shown in FIG. 1, it was confirmed that the effect of treatment with the Cannabis sativa extract (CE) (MT1) was similar to the effect of treatment with 1,000 μg/ml of metformin (MET), and the target DNA-binding activity further decreased when the cells were treated with each of the extract mixtures (MT2 to MT6).

Thereby, it could be confirmed that the extract mixtures (MT2 to MT6) of the present disclosure inhibited the target DNA-binding activity of the lipogenic transcription factor SREBP-1c and showed stronger inhibitory activity than metformin.

### Test Example 4: Effect of Reducing Body Weight Gain Caused by High-Fat Diet Therapy

High-Fat-Diet Therapy Test Groups

Each of the Cannabis sativa extract (CE) (MT1) and extract mixtures (MT2 to MT6) of the present disclosure and phloretin was dissolved in 0.5% DMSO, and then was administered orally to each mouse of each test group at a dose of 20 mg/kg every other day for 14 weeks. Another mouse group was administered DMSO in the same manner.

The body weight of each mouse was measured in units of 0.01 g at the same time every day, and the food intake was measured once a week (every 7 days).

After 12 weeks of administration of the high-fat diet, adipose tissue was isolated from each mouse and weighed.

Standard Diet Therapy Test Group

The remaining one mouse group was administered DMSO in the same manner using standard diet therapy at the same temperature under the same environmental conditions as the above-described high-fat diet therapy, and then the body weight of each mouse was measured. After 12 weeks of administration of the diet, adipose tissue of each mouse was isolated, and the size and weight thereof were measured and used as a negative control.

Isolation of Adipose Tissue

The adipose tissues isolated from the high-fat-diet therapy test groups and the standard diet therapy test group were subjected to histological examination using a hematoxylin and eosin (H&E) staining method.

Specifically, each adipose tissue was embedded in paraffin, frozen, sectioned to a thickness of 8 μm using a cryocut microtome, and then mounted on a slide glass. Each of the slides having the section mounted thereon was deparaffinized by 5 minutes of immersion in xylene, and hydrated using ethanol at gradually decreasing concentrations (100%-95%-85%-70% for 2 minutes each).

Thereafter, each slide was washed with water to remove the remaining ethanol, and stained with hematoxylin for 6 minutes. Then, each slide was immersed in and taken out of a mixed solution of 1% hydrochloride-ethanol (HCl-EtOH), and this process was repeated three times so that the hematoxylin was sufficiently absorbed into the tissue. Then, the slide was immersed in and taken out of 0.5% ammonia water, and this process was repeated 10 times, thereby fixing the stain.

The tissue section stained with hematoxylin was stained again with eosin for 1 minute and dehydrated using ethanol at increasing concentrations (70%-85%-95%-100% for 2 minutes each).

Effect on Weight Loss

Each of the dehydrated tissue slides was washed clean by 5 minutes of immersion in xylene, and then completely dried at room temperature. Then the section of the tissue was observed under a microscope, and the body weight was measured.

As a result, as shown in FIG. 2, it was confirmed that, among the mouse groups to which the high-fat-diet therapy was applied, the mouse groups to which the Cannabis sativa extract (CE) (MT1) and extract mixtures (MT2 to MT6) of the present disclosure were administered, respectively, showed a significant decrease in weight gain compared to the mouse groups to which DMSO and phloretin were administered, respectively.

This suggests that administration of each of the Cannabis sativa extract (CE) (MT1) and extract mixtures (MT2 to MT6) of the present disclosure has the effect of suppressing weight gain. It was confirmed that this effect of suppressing weight gain was not an effect attributable to a difference in food intake, from the fact that there was no difference in food intake between the test groups.

### Test Example 5: Effects of Cannabis sativa Extract on Blood Glucose, Triglyceride and Cholesterol Levels

Effect on Blood Glucose and Triglyceride Levels

In order to examine the effect of each of the Cannabis sativa extract (CE) (MT1) and extract mixtures (MT2 to MT6) of the present disclosure, confirmed in Test Examples 2 to 4, on a living body in a high-fat diet-induced obesity mouse model, the following experiment was performed.

The collected blood was clotted and then centrifuged at 8000 rpm for 10 minutes, and the serum was collected. The levels of glucose and triglyceride in the serum were measured using a blood biochemical analyzer (Modular analytics, Hitachi, Japan).

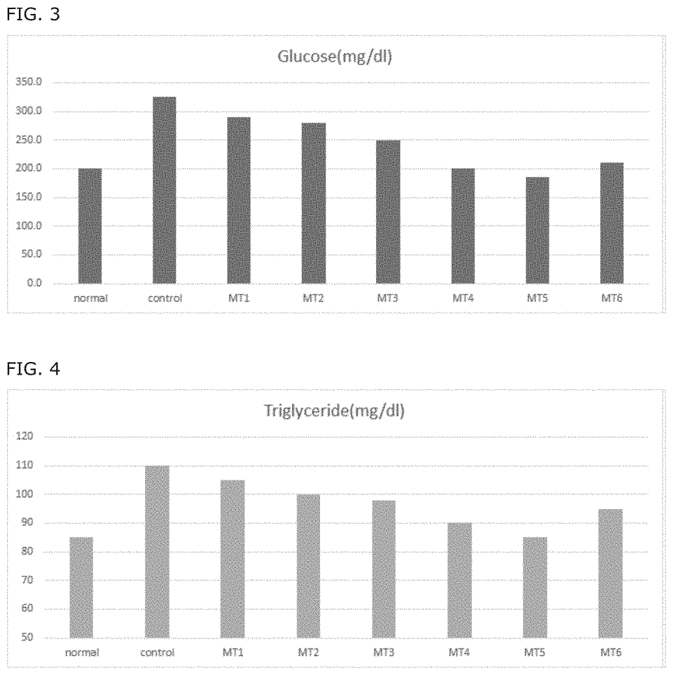

As a result, as shown in FIGS. 3 and 4, it was confirmed that the serum glucose level (FIG. 3) and the serum triglyceride level (FIG. 4) increased in the control group compared to the normal group, but the serum triglyceride level concentration-dependently decreased in the groups treated with each of the Cannabis sativa extract (CE) (MT1) and extract mixtures (MT2 to MT6).

Effect on Cholesterol Level

The collected blood was clotted and then centrifuged at 8000 rpm for 10 minutes, and the serum was collected. The levels of LDL-cholesterol and total cholesterol in the serum were measured using a blood biochemical analyzer (Modular analytics, Hitachi, Japan).

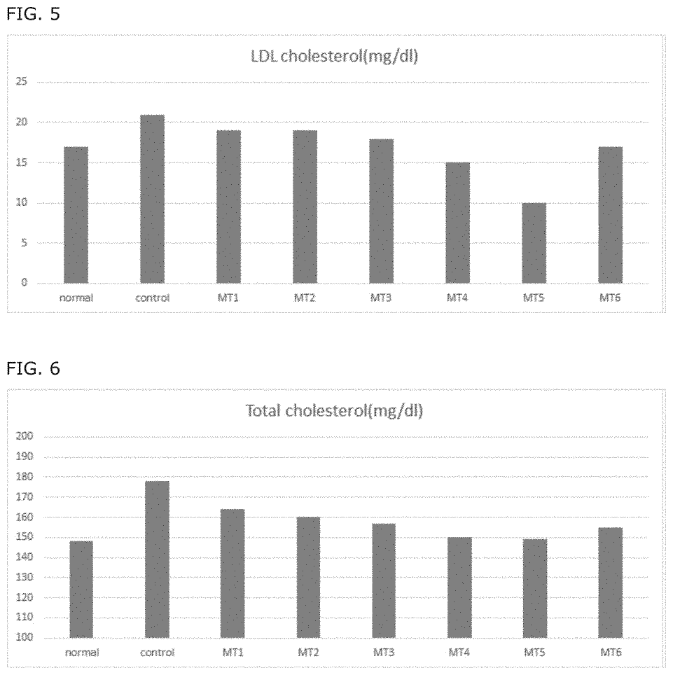

As a result, as shown in FIGS. 5 and 6, it was confirmed that the serum LDL-cholesterol level (FIG. 5) and the serum total cholesterol level (FIG. 6) increased in the control group compared to the normal group, but the serum LDL-cholesterol level and the serum total cholesterol level concentration-dependently decreased in the groups treated with each of the Cannabis sativa extract (CE) (MT1) and extract mixtures (MT2 to MT6).

Thereby, it could be confirmed that each of the Cannabis sativa extract (CE) (MT1) and extract mixtures (MT2 to MT6) had the effect of treating metabolic diseases, such as obesity, diabetes, hypertriglyceridemia and hypercholesterolemia, by reducing body weight, adipose tissue and liver weight and lowering the levels of glucose, triglyceride, LDL cholesterol and total cholesterol in blood.

### Test Example 6: Palatability Test

Tea beverages were prepared by diluting each of the Cannabis sativa extract (CE) (MT1) and extract mixtures (MT2 to MT6) of the present disclosure. Each of the tea beverages were tasted by 10 panelists, and the taste and flavor thereof were scored on a 10-point scale (1 to 10). The average values of the scores (any fraction of 0.5 or more is rounded up to the next higher whole number) are shown in Table 3 below. In the scores in

Table 3 below, a higher score indicates higher palatability.

TABLE 3

| | MT1 | MT2 | MT3 | MT4 | MT5 | MT6 |
|---|---|---|---|---|---|---|
| Taste | 6.0 | 6.0 | 6.5 | 7.0 | 7.5 | 6.0 |
| Flavor | 6.0 | 6.5 | 6.5 | 7.0 | 7.5 | 7.0 |
| Overall palatability | 6.0 | 6.0 | 7.0 | 7.0 | 7.5 | 6.5 |
| (average) | | | | | | |

(unit: score)

Referring to Table 3 above, it can be seen that, in the case of MT1 composed of the Cannabis sativa extract (CE) alone, the palatability was lowered due to the unique taste and flavor of the Cannabis sativa extract, and in the case of the mixtures MT2 to MT6, the palatability increased while the unique taste and flavor of the Cannabis sativa extract were neutralized by the other extracts.

In particular, it was confirmed that, in the case of MT3 to MT5, the effect of preventing and treating metabolic disease was excellent, and the palatability greatly increased while the taste and flavor were highly evaluated.

Therefore, each of the extract mixtures MT3 to MT5 according to the present disclosure may provide a functional food having an excellent effect on the prevention and treatment of metabolic disease while having higher flavor and taste palatability.

As described above, the present disclosure may provide a composition for preventing and treating metabolic disease, which contains an extract of the natural product Cannabis sativa as an active ingredient, and thus has little or no side effects when taken or administered, and has an excellent effect of preventing or treating metabolic syndrome by reducing body weight, adipose tissue, blood glucose, triglyceride and cholesterol levels through promotion of AMPK activity and inhibition of the activity of the lipogenic transcription factor SREBP-1c.

Although the preferred embodiments of the present disclosure have been described in detail above, the scope of the present disclosure is not limited thereto, and various modified and improved forms made by those skilled in the art on the basis of the basic concept of the present disclosure defined in the appended claims also fall within the scope of the present disclosure.

What is claimed is:

1. A composition for preventing and treating metabolic disease containing a Cannabis sativa extract as an active ingredient.

2. The composition of claim 1, wherein the Cannabis sativa extract contains cannabidiol and terpene.

3. The composition of claim 1, wherein the metabolic disease includes a disease selected from the group consisting of obesity, diabetes, hyperlipidemia, hypertension, hypercholesterolemia, hyperinsulinemia, arteriosclerosis and fatty liver.

4. The composition of claim 1, wherein the composition inhibits an activity of a lipogenic transcription factor by promoting AMPK activity.

5. The composition of claim 4, wherein the lipogenic transcription factor is SREBP-1c (sterol regulatory element-binding protein-1c).

6. A food composition for preventing metabolic disease comprising the composition according to claim 1.

7. A pharmaceutical composition for treating metabolic disease comprising the composition according to claim 1.
